(19) United States
(12) Patent Application Publication
(10) Pub. No.: US 2011/0003700 A1
(43) Pub. Date: Jan. 6, 2011

(54) ELIMINATION OF CONTAMINANTS ASSOCIATED WITH NUCLEIC ACID AMPLIFICATION

(75) Inventor: Douglas Spencer Millar, New South Wales (AU)

Correspondence Address: KNOBBE MARTENS OLSON & BEAR LLP, 2040 MAIN STREET, FOURTEENTH FLOOR, IRVINE, CA, 92614, US

(73) Assignee: HUMAN GENETIC SIGNATURES PTY LTD., North Ryde, NSW (AU)

(21) Appl. No.: 12/747,483

(22) PCT Filed: Dec. 19, 2008
(86) PCT No.: PCT/AU2008/001891

§ 371 Date: Jul. 27, 2010

(30) Foreign Application Priority Data
Dec. 20, 2007 (AU) 2007907002

(51) Int. Cl. C40B 30/00; C12P 19/34; C12Q 1/68

(52) U.S. Cl. 506/7; 435/91.2; 435/6

(57) ABSTRACT

Use of a non-natural base with an enzyme capable of degrading a nucleic acid containing a non-natural base in an amplification reaction to eliminate carry-over contaminants in the amplification reaction.

### TECHNICAL FIELD

The present invention relates to a strategy to overcome potential carry-over contamination with amplicons in amplification reactions.

### BACKGROUND ART

Polymerase chain reaction (PCR), developed around 1985 (Saiko, R. K., Scharf, S., Faloona, F., Mullis, K. B., Horn, G. T., Erlich, H. A., Arnheim, N. (1985). Science. 230: 1350-1354), has revolutionized the study of the biological system by enabling the detection and exponential amplification of miniscule amounts of nucleic acids, whether DNA or RNA, from virtually any target organism. A typical PCR reaction contains a mixture of a thermophilic enzyme such as Taq DNA polymerase, magnesium ions (Mg²⁺) and four deoxy-nucleoside tri-phosphates (dNTP), deoxyadenine triphosphate (dATP), deoxyguanine (dGTP), deoxythymine (dTTP) and deoxycytosine (dCTP). In theory, one copy of a nucleic acid molecule can be amplified exponentially generating enough amplified material so that the products can simply be visualized by agarose gel electrophoresis. Thus, if 40 cycles of amplification were carried out, roughly 2×10¹² copies of target nucleic acid would be generated in a single reaction (approximately 2ⁿ copies, where n is the number of PCR cycles used).

Due to the large number of amplicons generated in the PCR, carry-over contamination is problematic if strategies to manage accidental release of the amplification products (herein referred to as amplicons) are not implemented. Since the advent of PCR, derivatives of this method, as well as new methods of nucleic acid amplification (for example, reverse transcriptase-PCR (RT-PCR), ligase chain reaction, isothermal amplification, rolling circle amplification) have been developed, all of which are susceptible to carry-over contaminations. The presence of carry-over contaminants is one reason for false-positive results. In a research laboratory setting, false-positive results, particularly as a result of carry-over contaminants, would nullify the data. The experiment would therefore have to be repeated at considerably increased cost and effort. In a clinical setting, a false-positive result that may or may not be associated with carry-over contamination could have serious consequences, particularly if results are used for determining correct drug regimes for patient management.

Carry-over contamination occurs as a result of the accidental or unknowing introduction of previously amplified target DNA into an assay. The contaminant may have been introduced into the assay as a result of poor laboratory practices, or as a result of contaminated laboratory equipment, disposable and non-disposable glassware, plasticware and reagents, as well as carry-over contaminations between tests and other environmental contaminants.

There are two aspects to achieving a contaminant-free result—(a) prevention of the contamination as the “first line of defense”; and (b) destroying the contaminant, should the need arise.

Methods that can be used to destroy PCR contaminants include (i) UV irradiation; (ii) chemical elimination with sodium hypochlorite, hydrochloric acid or hydroxylamine hydrochloride, or (iii) treatment with one or more enzyme(s). UV-irradiation, which is effective for eliminating DNA/RNA from PCR premixes, laboratory surfaces, consumables and equipments, induces oxidation of the nucleotides, resulting in single and double-strand breaks and formation of cyclobutane rings between adjacent pyrimidines. Pyrimidine dimers formed inhibits extension of the product by Taq polymerase. Sodium hypochlorite is a strong oxidizer and will also induce single and double-strand breaks in the nucleic acids. Hydroxylamine hydrochloride, a reducing agent, disrupt normal base-pairing is an effective post-PCR contamination control but is mutagenic. However, these methods are mainly limited to the decontamination of surfaces and vessels and are incompatible with the actual set up of PCR reaction pre-mixes.

Enzymatic treatment of nucleic acid targets is a third method for eliminating contaminants and has been shown to be compatible with nucleic acid amplification reactions. Enzymes used to destroy contaminants can include DNases, RNases or endonucleases/DNA repair enzymes that target specific nucleotides or nucleosides, for example Uracil DNA glycosylases. Whereas DNAses and RNAses are effective for removing nucleic acids and their amplification productions, there may be residual enzymatic activity following inactivation that would interfere with downstream applications such as sub-cloning. More importantly, the use of such enzymes are again incompatible with the set-up of PCR reaction mixes as target molecules as well as possible contaminates would both be destroyed.

Uracil DNA glycosylase (UDG)/Uracil-N-glycosylase (UNG) is perhaps the most well know endonuclease used to eliminate carry-over contaminants. In the amplification reaction, dTTP is substituted with dUTP, which is a target for UDG/UNG digestion. The enzymes removes uracil from the sugar backbone of single and double stranded DNA, creating an abasic site that thermostable enzymes such as, Thermus aquatius derived DNA polymerase (Taq DNA polymerase) is inefficient at by-passing, thus inhibiting nucleic acid amplification. This UDG/UNG-dUTP contamination management strategy is compatible with single tube nucleic acid amplification, thereby minimizing the chance for further contaminations arising from opening tubes. Specifically, the enzyme may be included in a PCR reaction pre-mix containing dUTP instead of dTTP. UDG/UNG will specifically degrade any amplification contaminants containing dUTP that have been introduced into the PCR reaction mix prior to amplification. The enzyme is then inactivated during the initial denaturation step of the PCR to prevent the degradation of new target amplicons This system has been adapted to prevent carry-over contamination in PCR and is commercially marketed in various amplification kits. This strategy, however, is incompatible with sodium bisulphite treated nucleic acids as the process of this modification deaminates cytosine residues to uracil via a uracil sulfonyl intermediary.

Sodium bisulphite modification is a widely used technique for the investigation of methylation status of DNA as cytosine residues are converted by the bisulphite reaction whereas 5 Methyl-cytosine is resistant to this chemical modification. Methylation of cytosine residues in the human genome has been shown to be vitally important in the regulation and control of gene expression in development and embryogenesis. Hypo- and hypermethylation of cytosines in cytosine-guanine (CG) rich promoters of tumour suppressor genes and oncogenes have been implicated in the process of carcinogenesis. The sodium bisulphite modification of DNA has greatly facilitated the study of the role that 5-Methyl-cytosine plays in oncogenesis, development and embryogenesis. However the bisulphite method itself is theoretically and realistically incompatible with UDG/UNG-dUTP contamination strategy as the uracil residue generated during the bisulphite modification process would be degraded along with any cross over contaminant.

However, a recently developed method has enabled the use of UDG in the elimination of carry-over contamination in PCR reactions containing sodium bisulphite-treated target DNA. A critical step in sodium bisulphite modification is the removal of the sulphonate group from the 6-sulfonyl uracil intermediary. Typically this removal occurs by subjecting the treated DNA to an alkali environment at high temperatures before amplification or further processing as DNA polymerase is extremely inefficient at amplifying DNA containing bulky adducts. In this method, the 6-sulfonyl uracil (termed “SafeBis DNA” by Epigenomics AG) is not immediately desulphonated after modification and desalting. The sulfonyl group in SafeBis DNA appears to afford protection against UDG digestion therefore the UDG/UNG-dUTP contamination management strategy mentioned above can be coupled to the PCR without degrading the target DNA. Following UDG/UNG treatment, the reaction is heated to approximately 95° C. for between 20 and 30 minutes so that the UDG/UNG can be inactivated while simultaneously activating the Taq DNA polymerase and desulphonating the 6-sulfonyl uracil residues.

A number of limiting parameters are present with this method encompassing the SafeBis DNA. First, SafeBis DNA must be eluted and stored in a solution that is of neutral pH and at low temperature. Alkali pH of greater than 8-9 and/or high storage temperature will induce desulphonation of SafeBis DNA. The SafeBis method stipulates that the modified DNA is eluted with sterile water. It is recommended that for long term storage, DNA should be resuspended in TE buffer as DNA is vulnerable to acidic hydrolysis and therefore susceptible to degradation when stored in water.

Another limitation of this technique is that high contaminant concentrations may not be destroyed by this system. The removal of carry-over contaminant of UDG/UNG may not be optimal in the presence of high concentrations of contaminants. At PCR annealing temperatures of lower than 50° C., UDG may become re-activated, therefore digesting both the target nucleic acid and amplicons generated during the PCR. Importantly, SafeBis method has only been evaluated to successfully remove up to 10,000 copies of amplicons. The standard 40 cycles PCR, however, is able to amplify nucleic acid targets of approximately 2⁴⁰ or 2×10¹² copies. While UDG/UNG-dUTP carry-over contamination management strategy has been validated (to some extent) for SafeBis DNA, another effective method that does not exploit the uracil intermediary of sodium bisulphite conversion and the uracil-D- and uracil-N-glycolyase activity could overcome these limitations.

Other limitations of the conventional method, in a general sense, are associated with the properties of UDG/UNG enzymes. UDG is purportedly inactivated during the initial denaturation step of the PCR; denaturation at 95° C. for 10 mins is required to inactivate the enzyme. A standard PCR not utilizing hot-start Taq polymerase enzyme typically has a three to five minute initial denaturation at 95 degrees, which may not be adequate for inactivating UDG or UNG. In addition, heat stable UNG may retain some residual activity a temperatures of 75° C.-90° C. and UDG activity can be partially re-activated at temperatures of less than 55° C. In fact, it has been recommended that a 72° C. soaking/storage step should be included at completion of the PCR to ensure that the enzyme will remain inactive. A significant proportion of primers/oligonucleotides designed have optimal anneal temperatures of less than or around 55° C. thus newly amplified DNA strands may be cleaved during PCR. Part or all of these problems may be overcome by using a heat-labile UDG or UNG such as the HK™ UNG Thermolabile uracil-N-glycosylase.

The present inventor has developed a procedure that abrogates the need for UDG/UNG in carry-over contamination elimination in an amplification reaction.

### DISCLOSURE OF INVENTION

The present invention relates to a strategy for eliminating carry-over contaminants that are an unwanted product of nucleic acid amplification. Generally, the invention relates to the incorporation of a non-natural base into contaminant amplicons and the use of an enzyme capable of degrading a nucleic acid containing a non-natural base.

In a first aspect, the present invention provides use of a non-natural base with an enzyme capable of degrading a nucleic acid containing the non-natural base in an amplification reaction to eliminate carry-over contaminants.

The non-natural base is defined as a compound capable of being incorporated into nucleic acid and which is an endonuclease substrate, preferably Endonuclease V substrate. Examples of suitable non-natural bases are inosine, xanthosine, oxanosine, deoxynucleotide or deoxy-triphosphate analogues thereof. It will be appreciated that other non-natural bases may also be suitable for the present invention using the selective degrading characteristics of suitable endonucleases.

Preferably the enzyme capable of degrading a nucleic acid containing the non-natural base is an Endonuclease V.

The invention can be used in conjunction with the linear or exponential replication of normal and bisulphite treated nucleic acid such as DNA and RNA in vitro. In addition to the normal reaction conditions used in the amplification/replication protocols, adjustments can be made to the reaction conditions.

In a second aspect, the present invention provides an amplification reaction mixture comprising:

(a) deoxyinosine triphosphate (dITP) or deoxyxanthosine triphosphate (dXTP) or deoxyoxanosine (dOTP) or combinations thereof;

(b) deoxynucleotides (dNTPs) including deoxyguanine triphosphate (dGTP) deoxyadenine triphosphate (dATP), deoxycytosine triphosphate (dCTP), deoxythymine triphosphate (dTTP);

(c) an enzyme capable of degrading a nucleic acid containing a non-natural base; and

(d) thermostable polymerase.

Preferably, the amplification reaction mixture contains a limiting concentration of one or more of the dNTPs compared with the concentration of dITP, or dXTP, or dOTP or combinations thereof being used.

When the non-natural base dITP is used, the amplification reaction mixture preferably contains a limiting concentration of dGTP.

Preferably, the enzyme capable of degrading a nucleic acid containing a non-natural base is an endonuclease such as Endonuclease V. Endonuclease V, also known as deoxyinosine 3′-endonuclease, is a DNA repair enzyme derived from the Escherichia coli bacterium that is able to preferentially recognize single and double-stranded nucleic acids with incorporated deoxyinosine from a background of standard dNTPs. More recently other Endonuclease V enzymes have been isolated from organisms such as Salmonella and Thermotoga maritima (TMA) which have been shown to have a similar substrate recognition as the original Escherichia coli enzyme The enzyme cleaves the nucleic acid strand preferentially containing the inosine but also nucleic acid containing xanthosine and oxanosine residues at the second phosphodiester bonds 3′ to the lesion, leaving a nick with 3′ hydroxyl and 5′ phosphoryl groups. In the presence of a repair protein, the nucleotide analogue would then be excised and repaired. Endonuclease V will also recognize deoxyuridine residues, DNA with abasic sites or urea, base mismatches, insertion/deletion mismatches, hairpin and unpaired loops, flaps and pseudo-Y structures, but at a significantly lower rate.

The thermostable polymerases suitable for use with amplification of all nucleic acids include, but are not limited to, thermophilic and mesophilic DNA polymerases (for example, Tag, Pfu, Tth, Tfl, Pfx, Pfx50™, Tko, Bst, Vent®, Deep Vent™, Phusion™, ABV, UlTima, DyNAzyme EXT™, Therminator, polκ, pol IV, Dbh, Dpo4 and Dpo4-like enzymes, DNA I, Klenow fragment of DNA I polymerase, Phi 29, T4 and T7 DNA polymerases), reverse transcriptases (for example, AMV RT, M-MuLV RT, ThermoX RT™, Thermoscript RT™, Superscript III), and endonucleases (for example, Endonuclease III, IV, V, VIII, T7 Endonuclease I) and mutants or chimeras thereof. Enzymes that have been shown to be compatible with inserting dNTP, predominantly dCTP, opposite dITP are Taq, Pfu, Tth and KOD from Thermococcus kodakaraensis KOD1 and a modified variant of Taq polymerase termed 5D4 which has been shown to incorporate inosine residues more efficiently than standard Taq polymerase.

A number of modified polymerases are disclosed in EP 18012113 which are potential candidates for use in the present invention or be further modified to develop or enhance amplification activity. The enzyme 5D4 defined in EP 18012113 has been found by the present inventor to be particularly suitable for amplifying inosine containing nucleic acids.

The amplification reaction mixture may further contain a primer or primer sets for amplification.

In a third aspect, the present invention provides a method for eliminating carry-over contaminations that may occur during nucleic acid amplification comprising:

(a) providing a sample containing a nucleic acid template to be amplified;
(b) providing primers, probes or oligonucleotides for an amplification reaction;
(c) providing an amplification mixture according to the second aspect of the present invention;
(d) carrying out an incubation reaction such that any amplicons containing a non-natural base in the reaction mixture are degraded by the enzyme capable of degrading a nucleic acid containing a non-natural base;
(e) heating the incubated reaction mixture at a temperature to inactivate the enzyme capable of degrading a nucleic acid containing a non-natural base; and
(f) carrying out an amplification reaction to amplify a desired product from the nucleic acid template.

The method may further comprise:

(g) further processing or analysing the amplified product.

The processing or analyzing may include determining the sequence, methylation status, size, length of the amplified product by any suitable means such as gel electrophoresis, hybridization, digestion, real-time amplification, array based approaches, RFLP analysis and variations of the amplified product.

The sample may include native and bisulphite modified DNA, RNA and cDNA or a combination of any of these nucleic acids.

Preferably, the incubating step (d) is from about 0° C. to about 70° C. More preferably, the heating is at about 37° C.

Incubating step (d) can typically be carried out for 1 second to about 90 minutes. The present inventor has found that incubating at about 37° C. for about 15 minutes works well for most PCR reactions.

Preferably, the heating step (e) is from about 70° C. to about 95° C. More preferably, the heating is at about 95° C. to ensure total inactivation of enzyme capable of degrading a nucleic acid containing inosine.

The amplification reaction (f) is preferably carried out in the usual manner such that the thermostable polymerase copies the nucleic acid template using primers, probes or oligonucleotides.

The present invention is particularly suitable for bisulphite treated nucleic acid to eliminate carry-over contamination of an amplification reaction.

Unlike prior art, the method according to the present invention provides a strategy that harnesses the ability of suitable enzymes to incorporate dITP during the nucleic acid reverse-transcription and/or amplification process. Primarily, the invention allows for the incorporation of dITP into the nascent synthetic nucleic acid strand during the process of nucleic acid amplification. In subsequent reverse-transcription and/or amplification reactions, the method exploits the ability of an Endonuclease V enzyme or other suitable enzymes to recognise and cleave any carry-over contaminants containing dITP in the reaction vessel prior to the initiation of the reverse transcription- and/or amplification-proper. While the method is particularly suitable for use with sodium-bisulphite treated nucleic acids, the method is, and has been shown to be, applicable for the carry-over contaminant elimination in all reverse-transcription and amplification reactions using native nucleic acids as template. This carry-over contamination prevention measure is adaptable to all techniques involved in reverse-transcribing and/or amplifying nucleic acids in a linear or exponential manner (for example PCR, RT-PCR and/or other DNA replication methods), that is conducted in a single or multiple reaction vessels.

Throughout this specification, unless the context requires otherwise, the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia before the priority date of each claim of this specification.

In order that the present invention may be more clearly understood, preferred embodiments will be described with reference to the following drawings and examples.

### BRIEF DESCRIPTION OF THE DRAWINGS

FIG. 1 shows results of PCR amplification using PCR reaction mix supplemented with various concentrations of deoxyinosine triphosphates (dITP) and deoxyguanine triphosphates (dGTP).

FIG. 2 shows results of PCR amplification using Endonuclease V enzymatic digestion of PCR products from FIG. 1.

FIG. 3 shows results of PCR amplification after Endonuclease V treatment of “contaminant”.

FIG. 4 shows results of 20 and 25 cycles of PCR amplification after Endonuclease V treatment of “contaminant”.

FIG. 5 shows results of PCR amplification showing effect of variable Endonuclease V concentration on elimination of the “contaminant”.

### MODE(S) FOR CARRYING OUT THE INVENTION

The present inventor has developed a procedure that abrogates the need for UDG/UNG in carry-over contamination elimination. Instead, the properties of endonuclease V and its preferred substrate dITP or other preferred substrates such as xanthosine and oxanosine or combinations thereof is exploited to overcome the various limitations associate with working with bisulphite treated DNA. Indeed the present invention is applicable to all types of nucleic acids (DNA, RNA, cDNA) and is applicable in both bisulphite treated and non-treated nucleic acid samples.

The present invention provides excellent carry-over contamination control in bisulphite modified nucleic acid. The present invention allows for the complete and specific elimination of carry-over contaminants. Unlike the SafeBis method, the present invention allows for the elution, partial or complete desulphonation and stable storage of the modified nucleic acid in a suitable alkali buffer that not only facilitates the process of desulphonation but also protect the nucleic acids against degradation during storage. The combination of this powerful contamination elimination strategy with the robust sodium bisulphite treatment method (U.S. Pat. No. 7,288,373) allows for the reliable and accurate assessment of methylation states or the specific and sensitive detection of microorganisms.

The present invention is applicable in other linear and exponential amplification of unmodified nucleic acid templates, including but not limited to PCR, RT-PCR, isothermal amplification, rolling circle amplification, whole genomic amplification and all methods involving the linear or exponential reverse transcription and/or amplification of nucleic acids. Moreover, the present invention provides for the use of two other endonucleases, Fpg and hOGG1, for which the substrate is not a naturally occurring nucleotide or nucleoside in the RNA or DNA. Both enzymes have been reported to oxidize purines, preferentially 8-oxoguanine, by targeting the first phosphodiester bond 5′ and 3′ of the lesion for cleavage. 8-oxoguanine is a mutagenic base byproduct of oxidative reaction. As it is unlikely to occur inherently, use of the nucleoside analogue should work as well as the present invention. In addition, both xanthosine and oxanosine are spontaneous deamination products of guanine which are also recognized by Endonuclease V enzymes derived from different bacterial sources. Thus these non-natural bases may also be useful for incorporating into PCR products the eliminate unwanted PCR cross-over contamination

It is understood that the components used in the present invention can be provided in the form of a kit for elimination of carry-over contaminations in all techniques involving reverse-transcription and/or amplification of all types of nucleic acids.

### Non Natural Bases

Non-natural base is defined herein as a compound capable of being incorporated into nucleic acid and which is an endonuclease substrate, preferably Endonuclease V substrate. Examples of suitable non-natural bases are inosine, xanthosine, oxanosine, deoxynucleotide or deoxy-triphosphate analogues thereof. It will be appreciated that other non-natural bases may also be suitable for the present invention using the selective degrading characteristics of suitable endonucleases.

### Enzymes

The enzyme capable of degrading a nucleic acid containing a non-natural base is endonuclease such as Endonuclease V. Endonuclease V, also known as deoxyinosine 3′-endonuclease, is a DNA repair enzyme derived from the Escherichia coli bacterium that is able to preferentially recognize single and double-stranded nucleic acids with incorporated deoxyinosine from a background of standard dNTPs. More recently other Endonuclease V enzymes have been isolated from organisms such as Salmonella and Thermotoga maritima (TMA) which have been shown to have a similar substrate recognition as the original Escherichia coli enzyme The enzyme cleaves the nucleic acid strand preferentially containing the inosine but also nucleic acid containing xanthosine and oxanosine residues at the second phosphodiester bonds 3′ to the lesion, leaving a nick with 3′ hydroxyl and 5′ phosphoryl groups. In the presence of a repair protein, the nucleotide analogue would then be excised and repaired. Endonuclease V will also recognize deoxyuridine residues, DNA with abasic sites or urea, base mismatches, insertion/deletion mismatches, hairpin and unpaired loops, flaps and pseudo-Y structures, but at a significantly lower rate.

The thermostable polymerases suitable for use with amplification of all nucleic acids include, but are not limited to, thermophilic and mesophilic DNA polymerases (for example, Taq, Pfu, Tth, Tfl, Pfx, Pfx50™, Tko, Bst, Vent®, Deep Vent, Phusion™, ABV, UlTima, DyNAzyme EXT™, Therminator, polκ, pol IV, Dbh, Dpo4 and Dpo4-like enzymes, DNA I, Klenow fragment of DNA I polymerase, Phi 29, T4 and T7 DNA polymerases), reverse transcriptases (for example, AMV RT, M-MuLV RT, ThermoX RT™, Thermoscript RT™, Superscript III), and endonucleases (for example, Endonuclease III, IV, V, VIII, T7 Endonuclease I) and mutants or chimeras thereof and a modified variant of Taq polymerase termed 5D4 which has been shown to incorporate inosine residues more efficiently than standard Taq polymerase.

Examples of other polymerase enzymes possibly suitable for use in the present invention maybe obtained using the modification methods disclosed in WO 99/02671, WO 00/40712, WO 02/22869, WO 03/044187, WO 05/045 and EP 18012113 (Medical Research Council).

A number of modified enzymes are disclosed in EP 18012113 which are potential candidates for use in the present invention or be further modified to develop or enhance activity on nucleic acid containing non-natural bases. Examples include enzymes designated 2F3, 1A10, 1A9, 2F12, 1C2, 2G6, 1A8, 2F11, 2H4, 2H9, 1B12, 2H2, 1C8, 2H10X, 3A10, 3B5, 3B6, 3B8, 3B10, 3C12. 3D1, 4D1 and 5D4. The enzyme 5D4 has been found by the present inventor to be particularly suitable for incorporating inosine into nucleic acids.

### Sample Preparation

The sample can be prepared from tissue, cells or can be any biological sample such as blood, urine, faeces, semen, cerebrospinal fluid, lavage, cells or tissue from sources such as brain, colon, urogenital, lung, renal, hematopoietic, breast, thymus, testis, ovary, uterus, tissues from embryonic or extra-embryonic linages, environmental samples, plants, microorganisms including bacteria, intracellular parasites, virus, fungi, protozoan, viroid and the like. Mammalian cell types suitable for treatment by the present invention are summarized in B. Alberts et al., 1989, The Molecular Biology of the Cell, 2^{nd} Edition, Garland Publishing Inc New York and London, pp 995-997.

The transcription and/or amplification of native and bisulphite modified target sequences from samples of human, animal, plant, bacterial, fungal and viral origin is meant to cover all life cycle stages, in all cells, tissues and organs from fertilization until 48 hours post mortem, as well as samples that may be derived from histological sources, such as microscope slides, samples embedded in blocks, or samples extracted from synthetic or natural surfaces or from liquids.

The analyses include the naturally occurring variation between cells, tissues and organs of healthy individuals, (health as defined by the WHO), as well as cells, tissues and organs from diseased individuals. Diseased in this sense includes all human diseases, afflictions, ailments and deviant conditions described or referred to in Harrison's Principles of Internal Medicine, 12th Edition, edited by Jean D Wilson et al., McGraw Hill Inc, and subsequent later editions; as well as all diseases, afflictions ailments and deviant conditions described in OMIM (Online Mendelian Inheritance in Man, www.ncbi.gov), but with emphases on the leading causes of death, namely, malignant neoplasms, (cancer), ischaemic heart disease, cerebrovascular disease, chronic obstructive pulmonary disease, pneumonia and influenza, diseases of arteries, (including atherosclerosis and aortic aneurysm), diabetes mellitus, and central nervous system diseases, together with socially debilitating conditions such as anxiety, stress related neuropsychiatric conditions and obesity, and all conditions arising from abnormal chromosome number or chromosome rearrangements, (aneuploidy involving autosomes as well as sex chromosomes, duplications, deficiencies, translocations and insertions), as well as similar abnormalities of the mitochondrial genomes.

The normal or diseased individuals may be from (i) populations of diverse ethnicity and evolutionary lineages; (ii) strains and geographical isolates; (iii) sub species; (iv) twins or higher order multiplets of the same or different sex; (v) individuals arising from normal methods of conjugation, artificial insemination, cloning by embryonic stem cell methods, or by nuclear transfer, (from somatic or germ line nuclei), or from the input or modification of mitochondrial or other cellular organelles; (vi) individuals deriving from transgenic knock-out, knock-in or knock-down methods, (either in vivo, ex vivo, or by any method in which gene activity is transiently or permanently altered, e.g., by RNAi, ribozyme, transposon activation, drug or small molecule methodologies, Peptide Nucleic Acid (PNA), Intercalating Nucleic Acid (INA), Altritol Nucleic Acid (ANA), Hexitol Nucleic Acid (HNA), Locked Nucleic Acid (LNA), Cyclohexanyl Nucleic Acid (CNA), and the like; or nucleic acid based conjugates, including but not restricted to Trojan peptides, or individuals at any stages of pregnancy, normal or ectopic.

The analyses also include native and modified DNA, cDNA or RNA from prokaryotic or eukaryotic organisms and viruses (or combinations thereof), that are associated with human diseases in extracellular or intracellular modes, for the purposes of diagnostics and disease state monitoring or determining, and therapeutically altering, in both normally varying and diseased systems, the changed parameters and underlying mechanisms of:

(i) genetic diseases;
(ii) non-genetic or epigenetic diseases caused by environmentally induced factors, be they of biological or non-biological origin, (environmental in this sense being taken to also include the environment within the organism itself, during all stages of pregnancy, or under conditions of fertility and infertility treatments);
(iii) predisposition to genetic or non genetic diseases, including effects brought about by the “prion” class of factors, by exposure to pressure changes and weightlessness, or by radiation effects;
(iv) Genetic and epigenetic (for example of 5-methylcytosine) changes in the processes of aging in all cell types, tissues, organ systems and biological networks, including age related depression, pain, neuropsychiatric and neurodegenerative conditions and pre- and post-menopausal conditions, (including reduced fertility; in both sexes);
(v) Genetic and epigenetic (for example of 5-methylcytosine) changes in cancer, (including changes in cells with abnormal karyotypes arising from DNA amplification, deletion, rearrangement, translocation and insertion events), and their variations or alterations in different cell cycle phenomena (including cell cycle effects on diurnal rhythms, photoperiod, sleep, memory, and “jet lag”;
(vi) Genetic and epigenetic (for example of 5-Methylcytosine) changes in metabolic networks defined in the broadest sense, from the zygote through embryogenesis, foetal development, birth, adolescence, adulthood and old age (including metabolic effects brought about by hypoxia, anoxia, radiation of any type, (be it ionizing or non ionizing, or arising from chemotherapeutic treatments, high altitude exposure radiation from nearby natural sources, such as rocks or from “fallout” from military or government sponsored activities), stress, or by imbalances between the mitochondrial, nuclear or organellar genomes;
(vii) Genetic and epigenetic (for example of 5-methylcytosine) alterations due to responses at the molecular, cellular, tissue, organ and whole organism levels to proteins, polypeptides, peptides, and DNA, RNA, PNA, INA, ANA, HNA, LNA, CNA, and the like, or peptide aptamers (including any with post translational additions, post translational cleavage products, post translational modifications (such as inteins and exeins, ubiquination and degradation products); proteins, polypeptides and peptides containing rare natural amino acids, as well as single rare amino acids such as D-serine involved in learning, brain growth and cell death; drugs, biopharmaceuticals, chemical entities (where the definitions of Chemical Entities and Biopharmaceuticals is that of G. Ashton, 2001, Nature Biotechnology 19, 307-3111)), metabolites, new salts, prodrugs, esters of existing compounds, vaccines, antigens, polyketides, non-ribosomal peptides, vitamins, and molecules from any natural source (such as the plant derived cyclopamine);
(viii) Genetic and epigenetic (for example of 5-methylcytosine) alterations due to responses at the molecular, cellular, tissue, organ and whole organism levels to RNA and DNA viruses be they single or double stranded, from external sources, or internally activated such as in endogenous transposons or retrotransposons, (SINES and LINES);
(ix) Genetic and epigenetic (for example of 5-methylcytosine) alterations due to responses at the molecular, cellular, tissue, organ and whole organism levels to reverse transcribed copies of RNA transcripts be they of genetic or non genetic origins, (or intron containing or not);
(x) Genetic and epigenetic (for example of 5-methylcytosine) alterations due to responses at the molecular, cellular, tissue, organ and whole organism levels to: (a) DNA, RNA, PNA (peptide nucleic acids), INA (intercalating nucleic acids), ANA, HNA, LNA (locked nucleic acids), CNA (by HNA is meant nucleic acids as for example described by Van Aetschot et al., 1995; by MNA is meant nucleic acids as described by Hossain et al, 1998. ANA refers to nucleic acids described by Allert et al, 1999. LNA may be any LNA molecule as described in WO 99/14226 (Exiqon), preferably, LNA is selected from the molecules depicted in the abstract of WO 99/14226. More preferably, LNA is a nucleic acid as described in Singh et al, 1998, Koshkin et al, 1998 or Obika et al., 1997. PNA refers to peptide nucleic acids as for example described by Nielsen et al, 1991), and the like (or DNA, RNA, PNA, INA, ANA, HNA, LNA, CNA, aptamers of any in all combinations); including DNA, RNA, PNA, INA, ANA, HNA, LNA, CNA, and the like molecules circulating in all fluids including blood and cerebrospinal fluid as well as maternal fluids before, during and after pregnancy (b) combinations of conjugated biomolecules that are chimeras of peptides and nucleic acids; or chimeras of natural molecules such as cholesterol moieties, hormones and nucleic acids; and
(xi) Genetic and epigenetic (for example of 5-methylcytosine) alterations due to responses of stem cells, (either in vivo, ex vivo or in association with novel environments or natural and synthetic substrates (or combinations thereof), from human and animal origin to any of the perturbations described in (i) to (x) above.

### Bisulphite Treatment of Nucleic Acid

Any suitable method for obtaining nucleic acid material can be used. Examples include, but are not limited to, commercially available DNA, RNA kits or reagents, workstation, standard cell lysis buffers containing protease reagents and organic extraction procedures, which are well known to those of skill in the art.

The method can be carried out in a reaction vessel. The reaction vessel can be any suitable vessel such as tube, plate, capillary tube, well, centrifuge tube, microfuge tube, slide, coverslip, bead, membrane or any suitable surface.

Generally, the alkali environment is provided to the sample by adding an alkali such as NaOH. If the nucleic acid material is RNA then heat is preferably used instead of alkali to produce single stranded material without secondary structure. The alkali environment is provided to denature double stranded nucleic acid molecules into a state where the molecules are readily reactive with the bisulphite reagent. It will be appreciated, however, that any other denaturation method such as heat treatment or other suitable alkali or denaturing agent could be added or used such as KOH and any other alkali.

Generally, the bisulphite reagent is sodium metabisulphite. The bisulphite reagent is used to cause sulphonation of cytosine bases to cytosine sulphonate followed by hydrolytic deamination of the cytosine sulphonate to uracil sulphonate. It will be appreciated, however, that any other suitable bisulphite reagent could be used such as sulphite or acetate ions (see Shapiro, R., DiFate, V., and Welcher, M, (1974) J. Am. Chem. Soc. 96: 906-912).

The incubation with the sulphonating reagent can be carried out at pH below 7 and at a temperature which favors the formation of the uracil sulphonate group. A pH below 7 is optimal for carrying out the sulphonation reaction, which converts the cytosine bases to cytosine sulphonate and subsequently to uracil sulphonate. However, the methods can be performed with the sulphonation reaction above pH 7, if desired.

The sulphonation reaction can be carried out in the presence of an additive capable of enhancing the bisulphite reaction. Examples of suitable additives include, but not limited to, quinol, urea, DTT and methoxyamine. Of these reagents, quinol is a reducing agent. Urea and methyoxyamine are agents added to improve the efficiency of the bisulphite reaction. In addition, DTT can be used in the reaction to prevent the degradation of RNA by endogenous RNases. It will be appreciated that other additives or agents can be provided to assist in the bisulphite reaction. The sulphonation reaction results in methylated cytosines in the nucleic acid sample remaining unchanged while unmethylated cytosines are converted to uracils.

Reaction conditions found to work well are as follows. The DNA, or other nucleic acids, to be treated is made up to a volume of 20 μl and denatured by incubating with 2.2 □l freshly prepared 3M sodium hydroxide (BDH AnalaR #10252.4X) solution for 15 minutes at 37° C. The concentration of sodium hydroxide and incubation times can be adjusted as necessary to ensure complete denaturation of the template nucleic acid. 220 μl of a freshly prepared solution of 3 M sodium metabisulphite (BDH AnalaR #10356.4D) pH 5.0 (the pH is adjusted by the addition of 10M sodium hydroxide (BDH AnalaR #10252.4X) along with 12 μl of a 100 mM quinol solution (BDH AnalaR #103122E) is added. The concentration of quinol added can be anything in the range of about 10 to 500 mM as determined experimentally. The solution is then vortexed and overlayed with 208 μl of mineral oil (Sigma molecular biology grade M-5904). The sample is then incubated at a suitable temperature and for sufficient time, to allow time for full bisulphite conversion, for example at 80° C. for 45 minutes. It is understood by those skilled in the art that the volumes, concentrations and incubation time and temperature described above can be varied so long as the reaction conditions are suitable for sulphonation of the nucleic acids.

The converted nucleic acids are then desalted either by use of a desalting column, such as Zymo-Spin I columns according to the manufacturer's instructions, or by precipitation. For precipitation, samples are diluted so that the salts inhibitory to subsequent reactions are not co-precipitated with the sulphonated nucleic acids. The salt concentration is diluted to less than about 1 M. Generally, the dilution step is carried out using water or buffer to reduce the salt concentration to below about 0.5M. For example, the salt concentration is generally diluted to less than about 1 mM to about 1 M, in particular, less than about 0.5 M, less than about 0.4 M, less than about 0.3 M, less than about 0.2 M, less than about 0.1 M, less than about 50 mM, less than about 20 mM, less than about 10 mM, or even less than about 1 mM, if desired. One skilled in the art can readily determine a suitable dilution that diminishes salt precipitation with the nucleic acids so that subsequent steps can be performed with minimal further clean up or manipulation of the nucleic acid sample. The dilution is generally carried out in water but can be carried out in any suitable buffer, for example Tris/EDTA or other biological buffers so long as the buffer does not precipitate significantly or cause the salt to precipitate significantly with the nucleic acids so as to inhibit subsequent reactions. Generally, precipitation is carried out using a precipitating agent such as an alcohol. An exemplary alcohol for precipitation of nucleic acids can be selected from isopropanol, ethanol or any other suitable alcohol.

The desulphonation step can be carried out by adjusting the pH of the precipitated treated nucleic acid up to about 12.5. Exposure to alkaline environments tends to promote strand breaks in apurinic sites in the DNA induced by the previous exposure to an acidic pH. Therefore, the alkaline pH treatment is minimized if strand breaks are to be avoided. This step can be carried out efficiently at around pH 10.5-11.5 with a suitable buffer or alkali reagent. Examples of suitable buffers or alkali reagents include buffers having a pH 7.0-12.5. It will be appreciated by persons skilled in the art that suitable buffers or alkali reagents can be selected from the vast range of known buffers and alkali reagents available.

Temperature ranges for the desulphonation step are room temperature to about 96° C. and times can vary from 2 minutes to 96 hours or longer depending on the conditions used. One skilled in the art can readily determine a suitable time and temperature for carrying out the desulphonation reaction. Temperatures below room temperature can also be used so long as the incubation time is increased to allow sufficient desulphonation. Thus, the incubation step can be carried out at about 10° C., about 20° C., about 22° C., about 25° C., about 30° C., about 35° C., about 37° C., about 40° C., about 45° C., about 50° C., about 55° C., about 60° C., about 65° C., about 70° C., about 75° C., about 80° C., about 85° C., about 90° C., about 95° C., and about 96° C. A particularly useful temperature for carrying out the desulphonation reaction is in the temperature range 75° C. to 95° C.

### Amplification

The present invention provides a method that is used in conjunction with the linear or exponential replication of normal and bisulphite treated nucleic acid such as DNA and RNA in vitro. In addition to the normal reaction conditions used in the amplification/replication protocols, adjustments are made to the reaction conditions. In a preferred form, the present invention allows for the inclusion in the amplification reaction of (i) various concentrations of deoxyinosine triphosphate (dITP), (ii) a limiting concentration of the deoxyguanine triphosphate (dGTP) in the reaction mixture without the need to change the remaining deoxynucleotide (dNTP) concentrations (i.e. dATP, dCTP, dTTP), and (iii) Endonuclease V.

Inosine, which is derived from adenine via an adenosine or inosine monophosphate (IMP) intermediary, is formed when a ribose ring (ribofuranose) is attached to the hypoxanthine molecule. It is commonly found in tRNAs and is an essential component involved in gene translation of wobble base-pairs. Its ribo- and deoxyribonucleoside derivatives, ITP and dITP, are able to form natural base-pairings with DNA and RNA, although the base-pairings formed are weaker than the Watson-Crick base-pairing. Deoxyinosine was shown to have a affinity in the dNTPs in the following order: dI:dC>dI:dA>dI:dG˜dI:dT although dCTP has been reported to the sole substituent opposite dITP when the dITP-DNA act as the template for the PCR. Conversely, it was reported that substitution of dITP for dGTP in a PCR prior to direct sequencing was able to successfully overcome compression artefacts caused by stacking of sequenced fragments as well as render stable hairpin structures accessible for nucleic acid amplification. Conversely, addition of dITP in a standard sequencing reaction appears to promote premature termination near regions high in secondary structures but this may be over-come by reducing initiation temperatures of the sequencing reaction from 90° C. to 70° C. Presumably, this is associated the fact that substitution of dITP reduces the strand separation temperature and primer annealing temperatures despite the ability of Taq Polymerase to tolerate high temperatures. During direct sequencing, incorporation of dITP in the sequencing reaction is observed to prematurely terminate the sequencing reaction and is not recommended for direct sequencing although the premature termination rates may be reduce by reducing the reaction temperature

Endonuclease V (NEB catalog #M0305), also known as deoxyinosine 3′-endonuclease, is a DNA repair enzyme derived from the Escherichia coli bacterium that is able to preferentially recognize single and double-stranded nucleic acids with incorporated deoxyinosine from a background of standard dNTPs. In addition, Endonuclease V derived from T. maritima (Fermentas catalogue#EN0141) or any other suitable Endonuclease V enzyme such as Salmonella Endonuclease V can be used in the reaction. The enzyme cleaves the nucleic acid strand containing the non-natural base such as inosine and also xanthosine and oxanosine residues at the second phosphodiester bonds 3′ to the lesion, leaving a nick with 3′ hydroxyl and 5′ phosphoryl groups. In the presence of a repair protein, the DNA would then be excised and repaired. Endonuclease V will also recognize DNA with abasic sites or urea, base mismatches, insertion/deletion mismatches, hairpin and unpaired loops, flaps and pseudo-Y structures, but at a significantly lower rate.

The enzymes of interest for use with amplification of all nucleic acids include, but are not limited to, thermophilic and mesophilic DNA polymerases (for example, Taq, Pfu, Tth, Tfl, Pfx, Pfx50□, Tko, Bst, Vent_{R}®, Deep Vent□, Phusion□, ABV, UlTima, DyNAzyme EXI□, Therminator, pol□, pol IV, Dbh, Dpo4 and Dpo4-like enzymes, DNA I, Klenow fragment of DNA I polymerase, Phi 29, T4 and T7 DNA polymerases), reverse transcriptases (for example, AMV RT, M-MuLV RT, ThermoX RT□, Thermoscript RT□, Superscript III), and endonucleases (for example, Endonuclease III, IV, V, VIII, T7 Endonuclease I) and mutants or chimeras thereof. Enzymes that have been shown to be compatible with inserting dNTP, predominantly dCTP, opposite dITP are Taq, Pfu, Tth and KOD from Thermococcus kodakaraensis KOD1 and a modified variant of Taq polymerase termed 5D4 which has been shown to incorporate inosine residues more efficiently than standard Taq polymerase.

### EXAMPLES

In order to demonstrate the present invention inosine has been used as a representative non-natural base suitable for the present invention.

### Methods and Reagents

Chemicals were obtained as follows: Ethanol from Aldrich (St. Louis Mo.; 200 proof E702-3); Isopropanol from Sigma (St. Louis Mo.; 99%+Sigma I-9516); Mineral oil from Sigma (M-5904); Quinol from BDH (AnalaR #103122E); Sodium acetate solution 3M from Sigma (S-7899); Sodium chloride from Sigma (ACS reagent S9888); and Sodium hydroxide from BDH (AnalaR #10252.4X); Sodium metabisulphite from BDH (AnalaR #10356); Diethyl ether from Sigma (St. Louis Mo.; 309958); Hexane from Sigma (St. Louis Mo.; 650420); Luria broth from Oxoid (Liverpool; CM0996B); Magnesium chloride from Sigma (St. Louis Mo.; 63069); Mineral oil from Sigma (M-5904); Potassium chloride from Sigma (St. Louis Mo.; 60142); Span 80 From Fluka (Buchs CH; 85548); Tetracycline hydrochloride from Sigma (St. Louis Mo.; T8032); Triton X-100 from Sigma (St. Louis Mo.; 93426); Trizma hydrochloride from Sigma (St. Louis Mo.; T5941); Tween 80 from Sigma (St. Louis Mo.; P8074).

Enzymes/Reagents were obtained as follows: dNTPs from Promega (Madison Wis.; C1145); Glycogen from Roche (Indianapolis Ind.; #10 901 393 001); DNA markers from Sigma (Direct load PCR low ladder 100-1000 bp, Sigma D-3687 and 100-10 Kb, Sigma D-7058); PCR master mix from Promega (Madison Wis.; #M7505); Endonuclease V from New England Biolabs (Beverly Mass.; #M0305), dITP from Fermentas (Cat##R1191),

Solutions were follows: (1) 10 mM Tris/0.1M EDTA, pH 7.0-12.5; (2) 3M NaOH (6 g in 50 ml water; BDH AnalaR #10252.4X); (3) 3M Metabisulphite (7.6 g in 20 ml water with 416 □l 10 N NaOH (BDH AnalaR #10356.4D); (4) 100 mM Quinol (0.55 g in 50 ml water; BDH AnalaR #103122E); (5) 50×TAE gel electrophoresis buffer (242 g Trizma base, 57.1 ml glacial acetic acid, 37.2 g EDTA and water to 1 l); (6) 5× Agarose gel loading buffer (1 ml 1% Bromophenol blue (Sigma B6131), 1 ml Xylene Cyanol (Sigma X-4126), 3.2 ml Glycerol (Sigma G6279), 8 μl 0.5M EDTA pH 8.0, 200 μl 50×TAE buffer and water to 10 ml); and (7) 1×Taq buffer (50 mM KCl, 10 mM Tris-HCl, pH 9.0, 0.1% Triton X-100, 1.5 mM MgCl₂).

### Bisulphite Treatment of Template DNA

An exemplary protocol for the bisulphite treatment of nucleic acids is set out below and was used to generate template nucleic acids for amplification or copying by the novel enzymes. This protocol successfully resulted in retaining substantially all DNA treated. It will be appreciated that the volumes or amounts of sample or reagents can be varied.

To 2 □g of nucleic acid in a volume of 20 □l, 2.2 □l of 3 M NaOH (6 g in 50 ml water, freshly made) was added. This step denatures the double stranded nucleic acid molecules into a single stranded form, since the bisulphite reagent preferably reacts with single stranded molecules. The mixture was incubated at 37° C. for 15 minutes. Incubation at temperatures above room temperature can be used to improve the efficiency of denaturation.

After the incubation, 220 □l 3M Sodium Metabisulphite (3.35 g in 4.68 ml water with 320 □l 10 N NaOH; BDH AnalaR #10356.4D; freshly made) and 12 □l of 100 mM Quinol (0.55 g in 50 ml water, BDH AnalaR #103122E; freshly made) were added in succession. Quinol is a reducing agent and helps to reduce oxidation of the reagents. Other reducing agents can also be used, for example, dithiothreitol (DTT), mercaptoethanol, quinone (hydroquinone), or other suitable reducing agents. Likewise, additives which enhance the reaction, such as methoxyamine or urea, may also be incorporated. The sample was overlaid with 200 □l of mineral oil which prevented evaporation and oxidation of the reagents, but is not essential. The sample was then incubated for 45 minutes at 80° C. Other temperatures from 25° C. to 90° C. may also be used with incubation lengths varying from 5 minutes to 8 hours, or longer.

After the treatment with Sodium Metabisulphite, the oil was removed, and 2 □l glycogen (20 mg/ml; Roche #10 901 393 001) or tRNA (Roche #10 109 495 001) were added if the nucleic acid concentration was low. These additives are optional and can be used to improve the yield of nucleic acid obtained by co-precipitating with the target nucleic acid especially when the nucleic acid was present at low concentrations. Typically, glycogen was used in the precipitation of DNA whereas tRNA was used as a co-precipitant with RNA, although other co-precipitants may also be used.

Bisulphite modified nucleic acids were then desalted by use of a desalting spin column such as Zymo-spin columns (Zymo # C1003) according to the manufacturer's instructions. Alternatively, the samples can be isopropanol precipitated as follows: 800 □l of water is added to the sample, mixed and then 1 ml isopropanol is added. The water or buffer reduces the concentration of the bisulphite salt in the reaction vessel to a level at which the salt will not precipitate along with the target nucleic acid of interest. The sample is mixed again and left at 4° C. for 60 minutes, although other temperatures and lengths of incubation can be used as long as it effectively results in precipitation of the nucleic acid. The sample is centrifuged at 15,000×g for 10-15 minutes at 4° C. and the pellet washed with 70% EtOH. This washing treatment removes any residual salts that precipitated with the nucleic acids.

The pellet is allowed to dry and then resuspended in a suitable volume of buffer or water, depending on the downstream application. If desulphonation is desired, re-suspension in TE buffer (10 mM Tris, 0.1 mM EDTA) pH 10.5 and incubation at 95° C. for 20 minutes has been found to be particularly effective for desulphonation of DNA samples. Buffers at pH 7.0-12.5 can also be used and the sample may be incubated at 37° C. to 95° C. for 1 min to 96 hr, as needed to facilitate desulphonation of the nucleic acid to a level that is acceptable by the user.

The method described above can be preceded by digestion with one or more restriction enzymes. Two independent restriction enzyme digests were set up of the same sample of DNA as described below. The enzymes selected for digestion are typically dependent upon the sequence to be amplified. For example, digest 2 μg genomic DNA with EcoRI in a 20 μl volume for 1 hr at 37° C. This step is used to digest the genomic DNA into smaller fragments which are more amenable to bisulphite conversion than genomic DNA. Sonication or physical forces can also be used to shear the DNA into smaller sized fragments. The intensity of sonication and the length of sonication is selected based on the desired size of DNA fragments. A separate digestion reaction was carried out, for example, by digesting 2 μg genomic DNA with HindIII as described above. These or other suitable restriction enzymes can be selected for pre-treatment digestion. The digested DNA is treated with metabisulfite as described above.

### Results

### Elimination of Carry-Over Contaminant and PCR Amplification

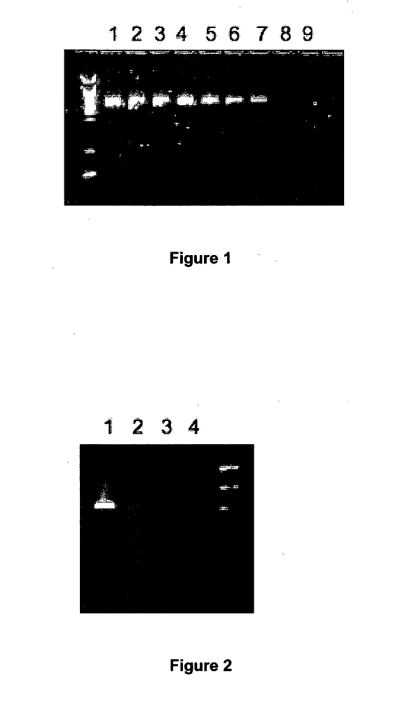

FIG. 1 shows results of PCR amplification using PCR reaction, mix supplemented with various concentrations of deoxyinosine triphosphates (dITP) and deoxyguanine triphosphates (dGTP).

One microliter of human genomic DNA (Promega, 20 ng/□l) was amplified in a final 25 □l reaction volume consisting of 1×PCR buffer, Taq DNA polymerase and 50 ng of each forward and reverse primers, MT-1F and MT-4R respectively, that are specific for mitochondrial gene, MARS. Two hundred micromoles of dATP, dTTP, dCTP were used in the PCR and the reaction was also supplemented with the following limiting amounts of dITP and dGTP.

Lane 1: 200 □M of dGTP and 0 □M of dITP, control reaction.
Lane 2: 180 □M of dGTP and 20 □M of dITP
Lane 3: 1600 □M of dGTP and 40 □M of dITP
Lane 4: 1200 □M of dGTP and 80 □M of dITP
Lane 5: 80 □M of dGTP and 120 □M of dITP
Lane 6: 40 □M of dGTP and 160 □M of dITP
Lane 7: 20 □M of dGTP and 180 □M of dITP
Lane 8: 0 □M of dGTP and 200 □M of dITP
Lane 9: 200 □M of dGTP and 0 □M of dITP, no template

The reaction was PCR amplified for 30 cycles at 95° C. for 20 seconds, 50° C. for 30 seconds and 65° C. for 30 seconds and products visualized by agarose gel electrophoresis. The results indicate that when dGTP was completely supplemented with dITP (Lane 8), no amplification products were detected. This indicates that dITP cannot completely substitute for dGTP in an amplification reaction.

FIG. 2 shows results of Endonuclease V enzymatic digestion of PCR products from FIG. 1.

Nine microliter of amplicons, previously amplified with MT-1F and MT-4R primers (see FIG. 1), were digested with 1 □l of Endonuclease V (10 U/□l). Samples were incubated at 37° C. for 30 minutes then inactivated at 95° C. for 5 minutes and 5 □l products visualized by agarose gel electrophoresis.

Samples used in the digestion were:

Lane 1: 200 □M of dGTP and 0 □M of dITP (control reaction)
Lane 2: 80 □M of dGTP and 120 □M of dITP
Lane 3: 40 □M of dGTP and 160 □M of dITP
Lane 4: 20 □M of dGTP and 180 □M of dITP

Ten units of Endonuclease V was shown to partially or completely digest amplicons generated using limiting amounts of dGTP and dITP. In contrast, the control reaction where only 200 □M of dGTP was used, remained undigested.

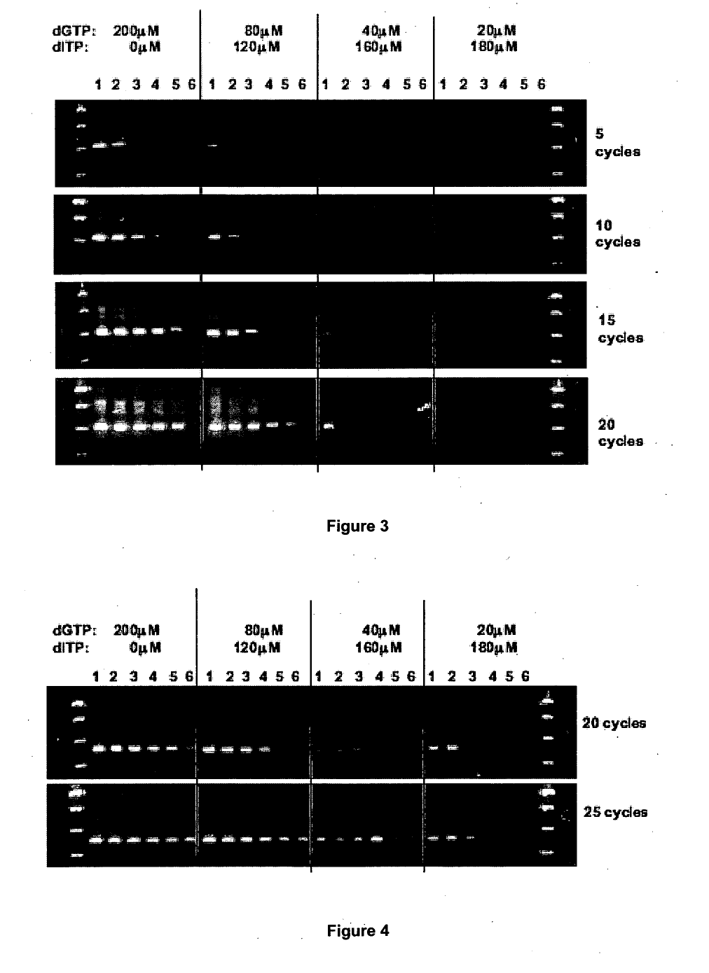

FIG. 3 shows results of PCR amplification after Endonuclease V treatment of “contaminant”.

Endonuclease V treated PCR products or “contaminants” from FIG. 2 were serially diluted. One microliter of the neat or serial diluted “contaminant” was amplified in a PCR reaction comprising of 1×PCR master mix (Promega cat# M7505), 50 ng of forward and reverse primers, MT-1F and MT-4R respectively.

The reaction was amplified for 5, 10, 15 and 20 cycles at 95° C. for 20 seconds, 50° C. for 30 seconds and 65° C. for 30 seconds and products visualized by agarose gel electrophoresis. At the completion of 5, 10, 15 and 20 cycles of PCR, the reaction was paused and the samples were soaked at 15° C. so that one set of samples may be removed. The PCR protocol was resumed when one set of samples was removed and visualized on an agarose gel.

The amount of contaminants amplified were:

Lane 1: Neat contaminant, undiluted
Lane 2: 1:10 dilution of contaminant
Lane 3: 1:100 dilution of contaminant
Lane 4: 1:1000 dilution of contaminant
Lane 5: 1:10000 dilution of contaminant
Lane 6: No template control

FIG. 4 shows results of 20 and 25 cycles of PCR amplification after Endonuclease V treatment of “contaminant”.

Endonuclease V treated PCR products or “contaminants” from FIG. 2 were serially diluted. One microliter of the neat or serial diluted “contaminant” was amplified in a PCR reaction comprising of 1×PCR master mix (Promega), 50 ng of forward and reverse primers, MT-1F and MT-4R respectively. The reaction was amplified for 20 or 25 cycles at 95° C. for 20 seconds, 50° C. for 30 seconds and 65° C. for 30 seconds and products visualized by agarose gel electrophoresis. Unlike FIG. 3, the PCR reaction was uninterrupted.

The amount of contaminants amplified were:

Lane 1: Neat contaminant, undiluted
Lane 2: 1:10 dilution of contaminant
Lane 3: 1:100 dilution of contaminant
Lane 4: 1:1000 dilution of contaminant
Lane 5: 1:10000 dilution of contaminant
Lane 6: 1:100000 dilution of contaminant

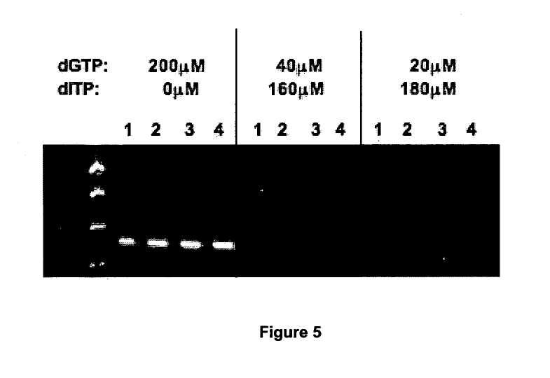

FIG. 5 shows effect of variable Endonuclease V concentration on elimination of the “contaminant”.

One microliter of human genomic DNA (Promega, 20 ng/□l) was amplified in a final 25 □l reaction volume consisting of 1×PCR buffer, Taq DNA polymerase and 50 ng of each forward and reverse primers, MT-1F and MT-3R respectively, that are specific for mitochondrial gene, MARS. Two hundred micromoles of dATP, dTTP, dCTP were used in the PCR and the reaction was also supplemented with limiting amounts of dITP and dGTP.

The PCR products were treated with 10 units (1), 5 units (2), 2.5 units (3) and 1.25 units (4) of Endonuclease V at 37° C. for 15 minutes. One microliter of the neat or serial diluted “contaminant” was amplified in a PCR reaction comprising of 1×PCR master mix (Promega), 50 ng of forward and reverse primers, MT-1F and MT-3R respectively. The reaction was amplified for 5 cycles at 95° C. for 20 seconds, 50° C. for 30 seconds and 65° C. for 30 seconds and products visualized by agarose gel electrophoresis.

The results show that dITP can be efficiently incorporated during PCR amplification as long as there is still some residual dGTP in the nucleotide mix and that complete digestion of PCR products containing dITP can be achieved by the use of Endonuclease V (see FIGS. 1 and 2). In addition, as shown in FIG. 3 and FIG. 4 supplementing the reaction mix with dITP/dGTP at a concentration of 180 μM/20 μM results in total degradation of the PCR product as reamplification of the digested products as shown in FIG. 2 yields no PCR products after 20 cycles of amplification even when the amplicon was undiluted and subsequently re-amplified (see FIG. 3). FIG. 5 shows that it is possible to reduce the concentration of Endonuclease V in the amplification reaction and still achieve a significant reduction in amplification even when only 1.25 Units of enzyme are used.

### PCR Amplification

PCR premixes are set up containing all the required components such as primers, enzyme, buffer, dNTPs, Mg²⁺ and template DNA. In addition to the standard PCR reagents the reaction is supplemented with dITP and Endonuclease V. If during the set-up reaction the mix has been contaminated with amplicons from a previous reaction (which will contain dITP) this contaminant can be removed prior to the initiation of PCR by heating the reaction to 37° C. for around 15 minutes. This pre-incubation step will not affect the template DNA or the PCR primers as neither of these components contains dITP. dITP is only incorporated into amplified material. The next step was to inactivate the Endonuclease V so that it does not degrade the sample that is about to be amplified. The inactivation was carried out during the initial 95° C. for 3-minute denaturation step. After denaturation the PCR reaction was carried out in the standard way again producing a new amplicon that contains dITP, which can then be subsequently analysed by any suitable means.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. Use of non-natural base with an enzyme capable of degrading a nucleic acid containing a non-natural base in an amplification reaction to eliminate carry-over contaminants in the amplification reaction.

2. The use according to claim 1 wherein the non-natural base is inosine, xanthosine, oxanosine, deoxynucleotide or deoxy-triphosphate analogues thereof.

3. The use according to claim 2 wherein the non-natural base is deoxyinosine triphosphate (dITP), deoxyxanthosine triphosphate (dXTP) or deoxyoxanosine (dOTP).

4. The use according to claim 2 wherein non-natural base is inosine or deoxyinosine triphosphate (dITP).

5. The use according to any one of claims 1 to 4 wherein the enzyme is an endonuclease.

6. The use according to claim 5 wherein the endonuclease is Endonuclease V derived from a mesophilic or thermostable bacterium.

7. The use according to any one of claims 1 to 6 wherein the amplification reaction is linear or exponential replication of normal or bisulphite treated nucleic acid.

8. The use according to claim 7 wherein the nucleic acid is DNA or RNA.

9. An amplification reaction mixture comprising:
(a) deoxyinosine triphosphate (dITP), deoxyxanthosine triphosphate (dXTP) or deoxyoxanosine (dOTP), or combinations thereof;
(b) deoxynucleotides (dNTPs) including deoxyguanine triphosphate (dGTP) deoxyadenine triphosphate (dATP), deoxycytosine triphosphate (dCTP), and deoxythymine triphosphate (dTTP);
(c) an enzyme capable of degrading a nucleic acid containing inosine, xanthosine or oxanosine; and
(d) thermostable polymerase.

10. The amplification reaction mixture according to claim 9 comprising deoxyinosine triphosphate (dITP).

11. The reaction mixture according to claim 9 or 10 containing a limiting concentration of one or more of the dNTPs compared with concentration of dITP, dXTP or dOTP.

12. The reaction mixture according to any one of claims 9 to 11 wherein the enzyme capable of degrading a nucleic acid containing anon-natural base is an endonuclease.

13. The reaction mixture according to claim 12 wherein the enzyme is an Endonuclease V.

14. The reaction mixture according to any one of claims 9 to 13 wherein the thermostable polymerase is selected from the group consisting of thermophilic and mesophilic DNA polymerases reverse transcriptases, endonucleases mutants and chimeras thereof.

15. The reaction mixture according to claim 14 wherein the thermostable polymerase is selected from Taq, Pfu, Tth, 5D4 and KOD from Thermococcus kodakaraensis KOD1.

16. The reaction mixture according to any one of claims 9 to 15 further containing a primer or primer sets for amplification.

17. A method for eliminating carry-over contamination that may occur during nucleic acid amplification comprising:
providing a sample containing a nucleic acid template to be amplified;
providing primers, probes or oligonucleotides for an amplification reaction;
providing an amplification mixture according to any one of claims 9 to 15;
carrying out an incubation reaction such that any amplicons containing inosine, xanthosine, or oxanosine in the reaction mixture are degraded by the enzyme capable of degrading a nucleic acid containing inosine, xanthosine or oxanosine;
heating the incubated reaction mixture at a temperature to inactivate the enzyme capable of degrading a nucleic acid containing inosine, xanthosine or oxanosine; and
carrying out an amplification reaction to amplify a desired product from the nucleic acid template.

18. The method according to claim 17 further comprising:
processing or analysing the amplified product.

19. The method according to claim 18 wherein the processing or analyzing comprises determining the sequence, methylation status, size, length of the amplified product.

20. The method according to claim 19 wherein the processing or analyzing comprises gel electrophoresis, hybridization, digestion, real-time amplification, array based approaches, RFLP analysis of the amplified product.

21. The method according to any one of claims 17 to 20 wherein the sample comprises native and bisulphite modified DNA, RNA and cDNA or a combination thereof.

22. The method according to any one of claims 17 to 21 wherein the incubation reaction is carried out at a temperature from 0° C. to 70° C. for 1 second to 90 minutes.

23. The method according to claim 22 wherein the temperature is about 37° C. at about 15 minutes.

24. The method according to any one of claims 17 to 23 wherein the heating step is from 70° C. to 95° C.

25. The method according to any one of claims 17 to 24 wherein nucleic acid template is treated with bisulphite.
